(19) United States
(12) Patent Application Publication
(10) Pub. No.: US 2006/0015119 A1
(43) Pub. Date: Jan. 19, 2006

(54) Positioning system with cannulated implant

(76) Inventors: Norman Plassky, Erfurt (DE); Gregor Tuma, Munchen (DE)

Correspondence Address: RENNER, OTTO, BOISSELLE & SKLAR, LLP;Nineteenth Floor, 1621 Euclid Avenue, Cleveland, OH, 44115-2191, US

(21) Appl. No.: 11/181,018

(22) Filed: Jul. 13, 2005

Related U.S. Application Data
(60) Provisional application No. 60/590,221, filed on Jul. 22, 2004.

(30) Foreign Application Priority Data
Jul. 14, 2004 (EP) 04016593.8

(51) Int. Cl. A61F 2/34

(52) U.S. Cl. 606/99

(57) ABSTRACT

A positioning system for placing an implant into a cavity of a bone, comprising a cannulated implant that is substantially smaller in the implanting area than the cavity and a positioning device that can hold the implant in a settable position in the cavity.

### RELATED APPLICATION DATA

This application claims priority of U.S. Provisional Application No. 60/590,221 filed on Jul. 22, 2004, which is hereby incorporated herein by reference in its entirety.

### FIELD OF THE INVENTION

The present invention relates to a positioning system for placing an implant into a cavity of a bone. In particular, the present invention relates to a positioning system for a revision implant.

### BACKGROUND OF THE INVENTION

Different procedures currently exist for placing implants or constructing a bone-like structure using cement and other fillers. In most cases, bone cement is poured into an open or exposed bone channel, for example a femoral cavity, once the old implant has been removed. This can be achieved with the aid of cannulated rods, or by introducing the cement into the bone cavity in some other way. Sample implants are sometimes used to form a mold in accordance with the actual implant, which then is introduced once the cement has hardened. In other methods, a pin is inserted into the cement and used as a trajectory for clearing a new channel or cavity. Cannulae, which once used can be removed again, are used for re-constructing vertebrae, for example.

Established methods also include pressing an implant or revision implant into the bone cavity, which, for example, can be cleared using a navigated tool. In most cases, the implant then is embodied slightly larger than the cavity into which it is to be introduced, in order that a kind of “pressing fit” can be achieved that holds the implant securely in the cavity. In other cases, the implant is encased in adhesives, fillers or cement, which then are to be dispersed in the cavity. In both cases cited above, it is not possible to achieve a high precision when positioning the implant; in the latter case, it is not guaranteed that all the remaining cavities have been reliably filled.

Such previously used systems and procedures are known, for example, from the following publications: U.S. Pat. No. 6,142,998; U.S. Pat. No. 5,171,288; U.S. Pat. No. 5,849,014; U.S. Pat. No. 5,192,283; U.S. Pat. No. 6,270,502; U.S. Pat. No. 5,624,443; U.S. 2003/0083662 A1 and U.S. 2003/0105468 A1.

### SUMMARY OF THE INVENTION

The present invention relates to a positioning system for placing an implant into a cavity of a bone. In particular, the present invention enables the implant to be positioned very precisely, and minimizes or eliminates large forces on the surrounding bone. Further, the present invention allows the bone cavity to be completely filled.

In accordance with the present invention, the positioning system comprises a cannulated implant which can be substantially smaller in the implanting area than the cavity, and a positioning device that can hold the implant in a settable position in the cavity. In other words, a device is provided which comprises a setting system for the implant which allows the implant to be precisely placed and held in the bone cavity in the correct position according to biomechanical, anatomical and technical considerations. These considerations can be determined through pre-operative planning, before the filler, adhesive or cement is introduced into the cavity via the cannulae in the implant. The term “substantially smaller than the cavity”, as used herein, is defined as the outer dimensions of the implant being substantially smaller than the inner dimensions of the cavity and/or do not exceed these inner dimensions at a substantial number of points or over a substantial length in the implanting area. If this is the case, the implant can be precisely positioned, once it has been inserted into the bone cavity.

Advantages of the system in accordance with the invention include reduction or elimination of stress on the bone structure from within due to the implant. Further, using the cannulae to introduce the filler, adhesive or cement into the cavity ensures that substantially all of the cavities in the bone are filled. Since the position of the implant can be set in accordance with the invention, the optimum end position of the implant also can be achieved with high precision. Overall, the invention enables implants to be placed in fewer steps, since sample implants do not have to be placed, and navigated and/or unnavigated clearing processes are unnecessary.

In a preferred embodiment of the invention, a reference array is assigned to the implant. The reference array allows the implant and its relative position with respect to the cavity to be localized and tracked by means of a surgical navigation system. Directly navigating the implant leads to an improved and more immediate positional control than in previously proposed, navigated cavity clearing methods. Further, known navigation systems can be used; these can be active or passive systems (with emitting or reflecting reference markers and camera tracking systems), and both optical and magnetic or electromagnetic navigation may be considered. A navigation system which may be used, for example, is known from DE 196 39 615 C2. Positioning need not necessarily be monitored and controlled using a navigation system, but can also be performed with the aid of intra-operative x-raying, for example.

The positioning device can be arranged positionally fixed with respect to the bone on one side, and can hold the implant on the other side. In principle, the positioning device can be advantageously arranged between the bone and the implant, in order that the setting movements have a direct effect on positioning with respect to said bone (bone cavity). In such an embodiment, however, it also is possible to use a holding device attached to the bone, by means of which the positioning device can be arranged positionally fixed with respect to the bone. Such a holding device can be a kind of clamp or the like.

In specific cases, the positioning device will comprise mechanical or electromechanical setting devices which allow the implant to be longitudinally and/or transversely shifted and/or rotated in the cavity. Such setting devices can be of a known type, for example pivoting devices, spindle drives or other angle-adjustable or length-adjustable devices that can be fixed when the implant has assumed the desired position.

A reference array also can be assigned to the bone or to the holding device, which allows the bone and/or holding device to be localized and tracked by means of a surgical navigation system, in particular the same navigation system which localizes and tracks the implant. This enables a sound relative navigation of the implant with respect to the bone, i.e., directly with respect to the part comprising the cavity.

The reference arrays (on the implant or on the bone and/or holding device) can be arranged such that they can move but are or can be fixed; in the latter case, the visibility for an optical navigation system can be further improved by adjusting the reference array. In a preferred embodiment, the reference array and the implant form a pre-calibrated unit whose geometry is known and can be clearly assigned in the navigation system.

The implant can be a cannulated permanent or revision implant comprising an inlet and peripheral outlets for a fixing mass, wherein the inlet is a separately provided inlet or leads through the interface for receiving the implant head.

In the following, the invention is explained in more detail on the basis of a preferred embodiment. The features described therein can of course also be implemented as a method and with computer assistance, and can be applied individually or in any combination within the framework of the invention.

### BRIEF DESCRIPTION OF THE DRAWINGS

In the following, reference is made to the enclosed drawings.

FIG. 1 is a partial schematic view of a positioning system in accordance with the invention, being used in setting a femoral implant.

FIG. 2 is a perspective view of an implant which may be used with the system in accordance with the invention.

FIG. 3 is a perspective view of the implant's placement in the femur after the placing process.

FIG. 4 is a perspective view of the implant inserted into the bone cavity, in a view onto the cavity entrance.

### DETAILED DESCRIPTION

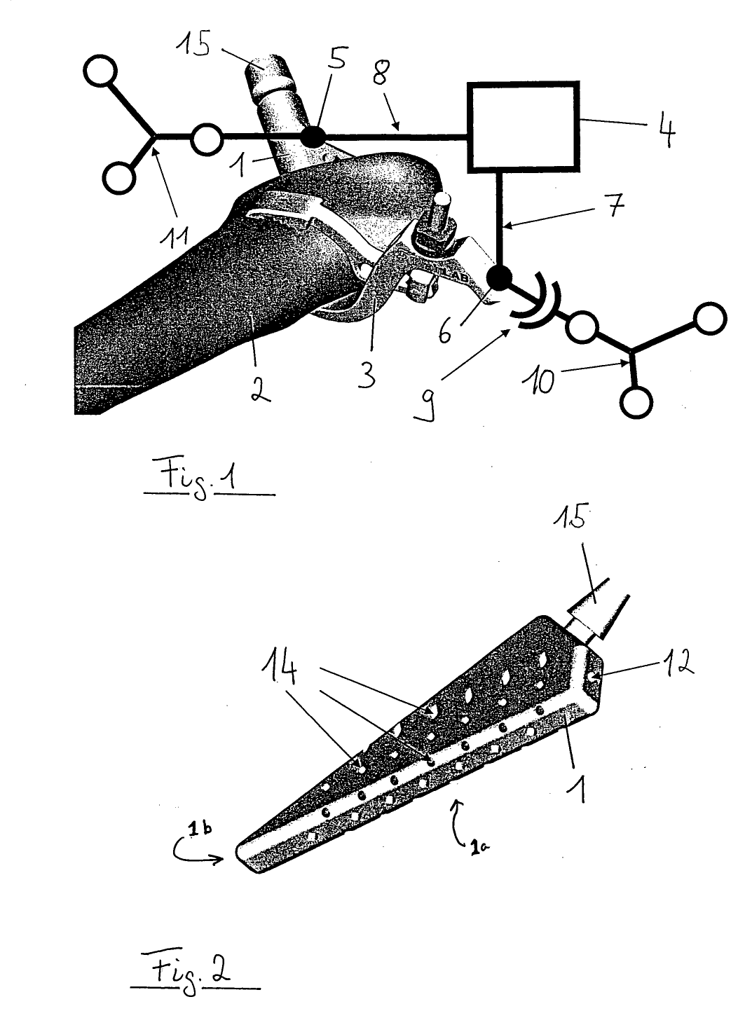

Identical parts are provided with identical reference numerals in the figures. In all the figures, the implant which may be used in accordance with the invention bears the reference numeral 1. The implant 1 is designed to be inserted into a cavity 13 (FIG. 4) of a femur 2 (FIGS. 1, 3 and 4), and is shown in more detail in FIG. 2. The implant 1 includes an implant body 1a tapering forward toward a distal end 1b and further including outlets 14 on its periphery for a fluid (filler, e.g., bone cement). Once the implant has been placed, the fluid is intended to fill the cavities between the implant 1 and the inner bone walls and fix the implant 1 with respect to the bone 2. The fluid is injected in via an inlet opening 12 of the implant 1, which is shown in FIGS. 2 and 4. An interface for receiving an implant head is indicated by the reference numeral 15. Embodiments in which the inlet opening leads through this interface are also conceivable.

In order to enable precise positioning of the implant 1, a setting device in accordance with the invention is used which is shown schematically in FIG. 1 and is designated by the reference numeral 4. As indicated above, the setting device 4 includes angle-setting devices, length-setting devices or rotation-setting devices, or a combination of such setting devices, and can be arranged positionally fixed with respect to the bone 2 by an arm 7; in this case via the arm 7 placed on a fixing point 6 of clamp 3, which is clamped to the bone 2. The position of the bone 2 can be localized and positionally tracked by a navigation system (not shown) via a reference star 10, which in turn is arranged on the clamp 3 such that it can be positionally adjusted via an adaptor 9. The reference star 10 can be positionally adjusted via an adaptor 9 in order to make it more visible in the navigation system, e.g, for the navigation cameras, and to fix it in an easily visible position.

On its other side, the positioning device 4 includes a setting arm 8, which is fixedly connected to the implant 1 at a fixing point 5. The implant 1 is shifted longitudinally or angularly or is rotated by the setting movements of the positioning device 4. The penetration depth, the varus/valgus rotation, the femoral antetorsion, the medial/lateral shift or the anterior/posterior tilt angle can be set. Adjustability in all degrees of freedom is in principle conceivable. Corresponding setting devices are known and need not be separately described here.

A reference star 11 is likewise rigidly connected to the implant 1 and enables the implant 1 to be localized and positionally tracked in the navigation system. In the present case, a rigid connection is shown since in a preferred embodiment of the invention, this is a pre-calibrated unit including the reference star 11 and the implant 1, the geometry of which is characteristic and known in the navigation system. It should be noted that the use of such pre-calibrated implants is not absolutely necessary within the framework of the invention. It is also possible to use non-pre-calibrated implants, to attach a reference star or other reference mechanism to the implant and to not calibrate the combination until they are in use. In such a case, a reference star, which, as in the case of the reference star 10, is arranged such that it can be moved but can also be fixed on the implant.

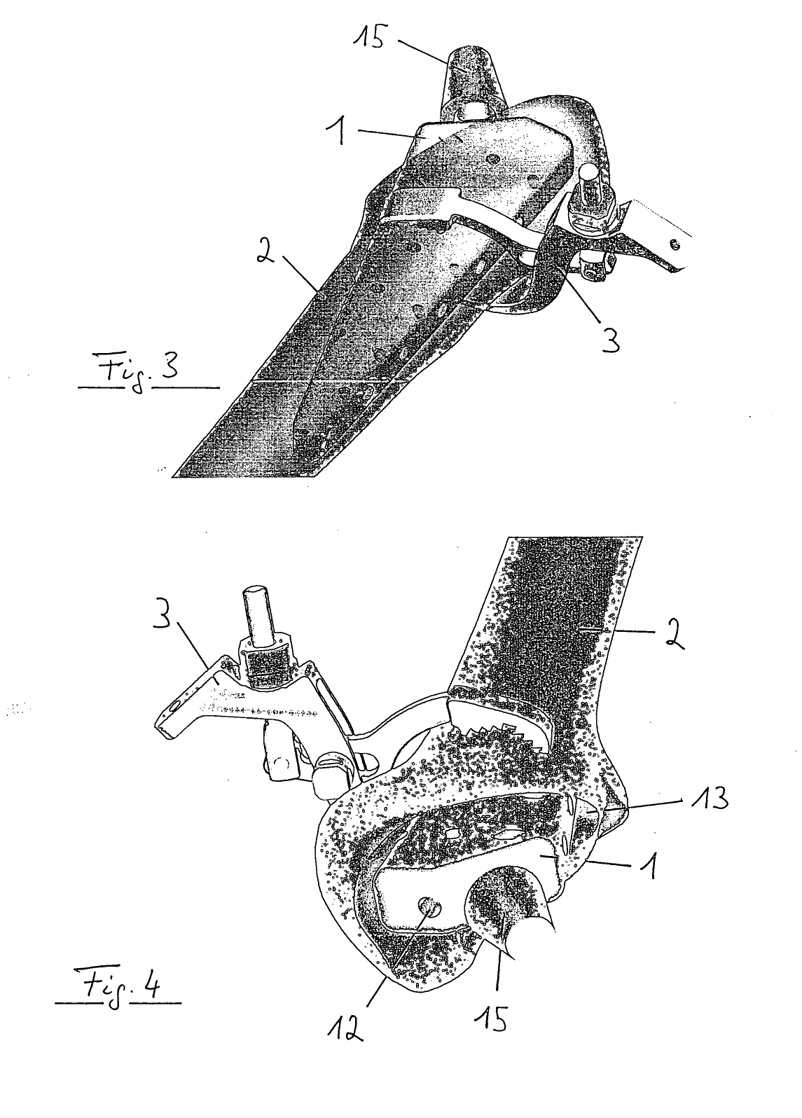

FIG. 3 shows how the implant comes to rest within the bone cavity. In addition, the bone wall is partially shown in transparent form so as to view the implant within the cavity 13. As follows from FIG. 4, the outer dimensions of the implant are smaller than the cavity 13 in the interior of the bone 2. Hence, the implant 1 can be inserted and optimally positioned in the cavity 13 without the bone structure being stressed or even damaged. Once the implant is optimally positioned, the remaining cavities are then filled by introducing the filler (bone cement) through the inlet 12 and the outlets 14.

In the following, use of the positioning system in accordance with the invention is also described on the basis of specific process in which an implant 1 is inserted into the cavity 13 of the bone 2. As a prerequisite, the patient's femoral channel is open or cleared, i.e., provided with the cavity 13. This femoral channel or cavity 13 can be the result of an old implant, already removed. Alternatively, the channel can be cleared, for example, in order to remove cancerous bone material. For performing and in order to assist the procedure, a navigation system is provided, and the reference star 10 is fixed to the bone 2 with the aid of the clamp 3 and the adaptor 9 in a position which is easily visible for the camera system of the navigational tracking means. It should be noted here that a reference star 10 could also be attached directly to the bone, for example to the distal end of the bone 2.

The bone is registered, i.e., positionally assigned, in the navigation system with the aid of the reference star 10. The reference star 11 then is arranged on the implant. If the system including the reference star 11 and the implant 1 is a pre-calibrated unit known to the navigation system, then it need not be registered therein; in other cases, registration can be performed here. Thereupon, the implant can then be inserted, roughly positioned using the reference star 11, into the cavity 13 of the bone 2, whereupon the positioning device 4 is attached both to the implant 1 (via the setting arm 8) and to the clamp 3 (via the arm 7).

With the aid of the positional information on the bone 2 and the implant 1 obtained from the navigation system, the position and orientation of the implant 1 in the cavity 13, i.e., with respect to the bone 2, can then be fine-adjusted. Adjustment can be performed, for example, by the setting movements of the positioning device 4 until the implant is situated precisely in the correct position, where it is then fixed. The correct position then can be aligned with the position determined beforehand as the optimum position by planning in the navigation system. If data on the bone and the geometry of the cavity are available in the navigation system, then the preciseness of the positioning can be determined for those points which are no longer visible once the implant has been introduced into the cavity 13, and corresponding correcting setting measures can be taken. Once the implant is placed in the correct position in the cavity 13, the positioning means 4 is fixed and the remaining cavity between the bone and the implant is filled with filler (bone cement) through the inlet 12 of the implant and the outlets 14, wherein the arrangement of the outlets 14 can ensure that the entire residual cavity is filled. Once the filler has then hardened, all the tools can be removed.

Although the invention has been shown and described with respect to a certain preferred embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

1. A positioning system for placing an implant into a cavity of a bone, comprising:
a cannulated implant, wherein the implant is substantially smaller in the implanting area than the cavity, and
a positioning device which can hold the implant in a settable position in the cavity.

2. The positioning system as set forth in claim 1, further comprising a first reference array assigned to the implant, said first reference array allowing the implant and a relative position of the implant with respect to the cavity to be localized and tracked by a surgical navigation system.

3. The positioning system as set forth in claim 2, further comprising a second reference array assigned to the bone, said second reference array allowing the bone to be localized and tracked by the surgical navigation system.

4. The positioning system as set forth in claim 2, wherein the first reference array is arranged fixedly on the implant or is arranged on the implant such that the first reference array can be moved and fixed.

5. The positioning system as set forth in claim 2, wherein the first reference array is arranged fixedly on the implant, and the implant is pre-calibrated for the navigation system.

6. The positioning system as set forth in claim 1, wherein the positioning device includes a first and second side, said positioning device arranged positionally fixed with respect to the bone on the first side, and the positioning device holds the implant on the second side.

7. The positioning system as set forth in claim 6, wherein the positioning device is arranged positionally fixed with respect to the bone by a holding device attached to the bone.

8. The positioning system as set forth in claim 7, wherein the holding device is a clamp.

9. The positioning system as set forth in claim 7, further comprising a reference array assigned to the holding device, said reference array allowing the holding device to be localized and tracked by a surgical navigation system.

10. The positioning system as set forth in claim 9, wherein the reference array is arranged fixedly on the holding device or is arranged on the holding device such that it can be moved and fixed.

11. The positioning system as set forth in claim 1, wherein the positioning device comprises a mechanical or electromechanical setting device which allows the implant to be longitudinally and/or transversely shifted and/or rotated in the cavity.

12. The positioning system as set forth in claim 1, wherein the implant comprises:
an implant head;
an inlet; and
peripheral outlets for a fixing mass, wherein the inlet is a separately provided inlet or leads through the interface for receiving the implant head.

13. The positioning system as set forth in claim 1, wherein the positioning system allows the implant and a relative position of the implant with respect to the cavity to be localized and tracked by intra-operative x-ray controlling.
